(19) United States
(12) Patent Application Publication
(10) Pub. No.: US 2020/0191789 A1
(43) Pub. Date: Jun. 18, 2020

(54) IN VITRO METHOD FOR DETECTING TUMOR GROWTH AND DIAGNOSING OR PROGNOSTICATING THE RISK OF METASTASIS IN A HUMAN SUBJECT THAT HAS BEEN DIAGNOSED WITH UVEAL MELANOMA

(71) Applicant: SERVIZO GALEGO DE SAÚDE, Santiago de Compostela (ES)

(72) Inventors: Antonio PIÑEIRO CES, Santiago de Compostela (ES); María José BLANCO TEIJEIRO, Santiago de Compostela (ES); María PARDO PÉREZ, Santiago de Compostela (ES)

(21) Appl. No.: 16/622,229

(22) PCT Filed: Jun. 13, 2018
(86) PCT No.: PCT/EP2018/065725

§ 371 Date: Dec. 12, 2019

(30) Foreign Application Priority Data
Jun. 14, 2017 (ES) P201730798

(51) Int. Cl. G01N 33/574; G01N 1/28

(52) U.S. Cl. CPC G01N 33/5743; G01N 1/28

(57) ABSTRACT

The present invention provides an in vitro method for detecting tumor growth and diagnosing or prognosticating the risk of metastasis in a human subject that has been diagnosed with uveal melanoma, wherein the method comprises using, as an indicator, the levels of PMEL/ME20-S/gp 100 positive exosomes, obtained from a biological sample isolated from the human subject selected from the list consisting of blood, or serum, of at least melanocyte protein PMEL/ME20-S/gp 100, and obtaining a result of the method by comparing the levels of at least said protein with a reference value or with the levels of a control, wherein an increase of melanocyte protein PMEL/ME20-S/gp 100 is indicative of a risk of the patient having or suffering from metastasis.

### FIELD OF THE INVENTION

The present invention refers to the medical field, in particular to the diagnostic field, more particularly to an in vitro method for detecting tumor growth and diagnosing or prognosticating the risk of metastasis in a human subject that has been diagnosed with uveal melanoma.

### BACKGROUND OF THE INVENTION

As the most common primary malignant intraocular tumor, uveal melanoma (UM) is also the main intraocular disease that can be fatal in adults. Its incidence in the general population is 5.3 to 10.9 cases per million people per year. Uveal melanoma disseminates mainly through the bloodstream and preferentially metastasizes to the liver. Even with successful treatment of primary UM tumors, patients remain at risk of developing metastases for more than 20 years after initial diagnosis. In the Collaborative Ocular Melanoma Study, Kaplan-Meier analysis estimated 2-, 5-, and 10-year metastasis rates of 10%, 25%, and 34%, respectively. However, only 0.24% of the patients exhibited detectable metastases at the time of diagnosis. In this regard, the metastatic rate has been related to the tumor height. Poor prognosis is associated with various clinical and molecular factors of the primary UM, such as tumor height, presence of monosomy 3, and gain of chromosome 8. More recently, UM research has evolved toward finding genetic prognostic markers to identify patients at risk for developing metastatic disease. In particular, tumor-specific mutations have been found in the GNAQ, GNA11, and BAP1 genes. In addition, gene expression profiling from fine-needle biopsies has emerged as a powerful tool for molecular prognostication in UM, able to discern low- and high-risk patients. However, the risk of underestimating the prognostic probability of metastasis and metastasis death by fine-needle aspiration biopsy has to be considered. Under this scenario, the identification of noninvasive blood biomarkers could have a crucial impact in the management of UM. Ideally, these prognostic markers would be effective for assessment of metastatic risk and guiding follow-up as well as facilitating adjuvant therapy decisions. We previously applied proteomics technology to detect UM tumor-specific proteins released into the extracellular surroundings and presumably to the blood circulation. We identified several potential UM biomarkers, including the 95-kDa pre-melanosome protein (PMEL), also known as glycoprotein 100 (gp100) and melanoma-associated ME20 (ME20M), and the oncoprotein PARK7/DJ-1. Both proteins, DJ-1 and the ME20M soluble form (ME20-S), were detected in the serum of patients with UM. Thereafter, a larger survey enabled us to describe for the first time that elevated serum levels of DJ-1 are associated with choroidal nevi transformation risk factors. ME20M is thought to be an oncofetal self-antigen that is normally expressed at low levels in quiescent adult melanocytes, but is overexpressed by proliferating neonatal melanocytes and during tumor growth. Because it is considered a tumor-associated antigen that is specific to patients with cutaneous melanoma, monoclonal antibodies against protein are routinely used in melanoma diagnosis. ME20M has a central role in melanosome biogenesis, mediating the maturation of melanosomes from stage Ito stage II.

Moreover, the secretion of the soluble form, ME20-S, has been suggested to protect melanoma cells from antibody-mediated immunity. Taking into account the melanoma-specific nature of this molecule, we hypothesize that it could be a good UM biomarker candidate.

In the present invention we demonstrate the identification of PMEL(ME20M) protein in exosomes shed by human uveal melanoma cells cultured in vitro. Since we have previously demonstrated the presence of circulating soluble form of PMEL (ME20-S) in patients with UM and its correlation with individual clinical data, we suggest exosomal-PMEL as a individual prognostic factor.

### BRIEF DESCRIPTION OF THE INVENTION

The present invention provides an in vitro method for detecting tumor growth and diagnosing or prognosticating the risk of metastasis in a human subject that has been diagnosed with uveal melanoma, wherein the method comprises using, as an indicator, the levels of PMEL/ME20-S/gp 100 positive exosomes, obtained from a biological sample isolated from the human subject selected from the list consisting of blood, or serum, of at least melanocyte protein PMEL/ME20-S/gp 100, and obtaining a result of the method by comparing the levels of at least said protein with a reference value or with the levels of a control, wherein an increase of melanocyte protein PMEL/ME20-S/gp 100 is indicative of a risk of the patient having or suffering from metastasis.

More particularly, the present invention provides an in vitro method for tumor growth assessment, and diagnosing or prognosticating the risk of metastasis in a human subject that has been diagnosed with uveal melanoma, wherein the biological sample is treated with a lysis agent capable of disrupting the membrane of the exosomes prior to determining the expression level of the melanocyte protein PMEL/ME20-S/gp 100.

More particularly, the present invention provides an in vitro method for diagnosing or prognosticating the risk of metastasis in a human subject that has been diagnosed with uveal melanoma, wherein the biological sample is treated so that the exosomal fraction of the melanoma tumour cells is obtained and the determination of the expression levels of PMEL/ME20-S/gp 100 is performed in such exosomal fraction, wherein such exosomal fraction is treated with a lysis agent capable of disrupting the membrane of the exosomes prior to determining the level of PMEL/ME20-S/gp 100.

### BRIEF DESCRIPTION OF THE DRAWINGS

FIG. 1 shows the applications of PMEL-exosomes analysis along UM development. PMEL-exosomes would show primary tumor growth, may differentiate low and high risk tumors, assessment of metastatic risk and guiding follow-up as well as facilitating adjuvant therapy decisions.

FIG. 2 shows the methodology to isolate and quantify PMEL in exosomes liberated from primary and metastatic UM tumor cells. Circulating exosomes would be purified by specific exosome antigens for further isolation of UM-PMEL positive exosomes by means of specific anti-PMEL antibodies.

FIG. 3 shows the proof of concept of this invention. Primary UM from enucleated eyes and metastatic tumor cells isolated from UM patients were cultured in vitro. The secretome derived from these cells in culture was ultracentrifuged following standard protocols to isolate UM microvesicles. Characterization of these vesicles confirmed their exosomal nature as tested by electron microscopy, light scattering and immunodetection of exosomes antigens. UM derived exosomes proteome was analyzed by mass spectrometry. Among other proteins, human PMEL was identified in UM secreted exosomes.

FIG. 4 shows 1. ME20-S circulating levels are statistically elevated in patients with primary UM tumors; 2. The ME20-S circulating levels positively correlate with tumor size; 3. ME20-S levels decrease to control levels in those patients were their primary tumor was treated by braquitherapy/enucleation (UM disease free patients); 4. ME20-S levels are statistically elevated in those patients with systemic disease. Therefore, in this invention we suggest the quantification of PMEL-positive exosomes liberated into the circulation as a specific UM biomarker to assay: a) primary tumor growth, b) tumor risk of metastasis, c) early systemic dissemination (before any scan) and d) systemic therapy follow up (chemotherapy/immunotherapy).

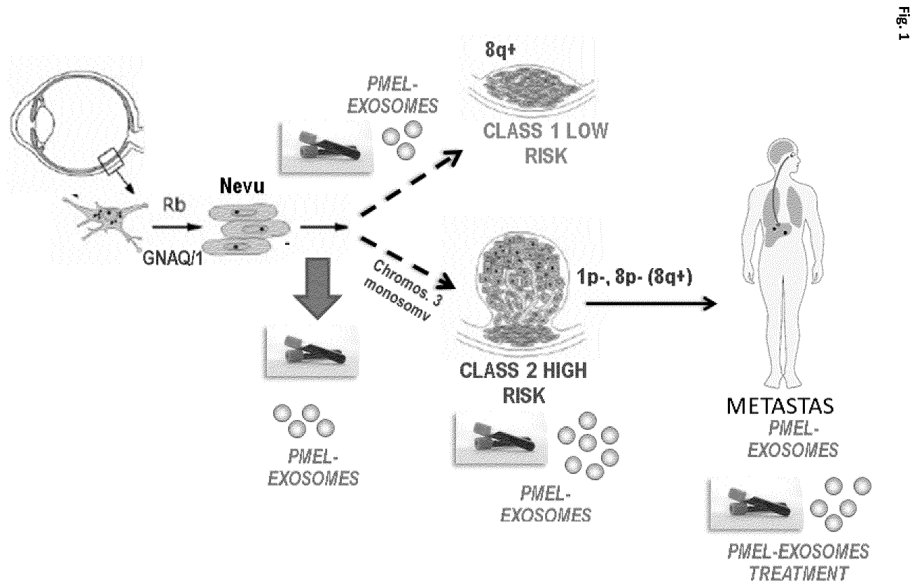

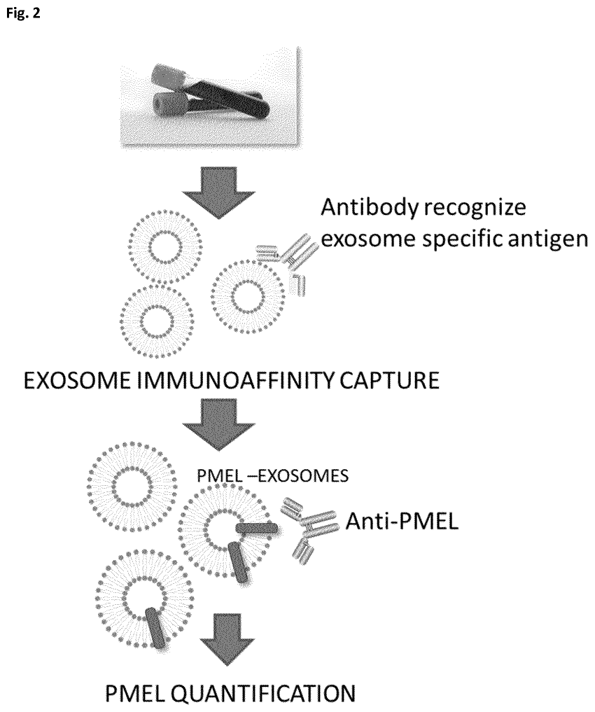

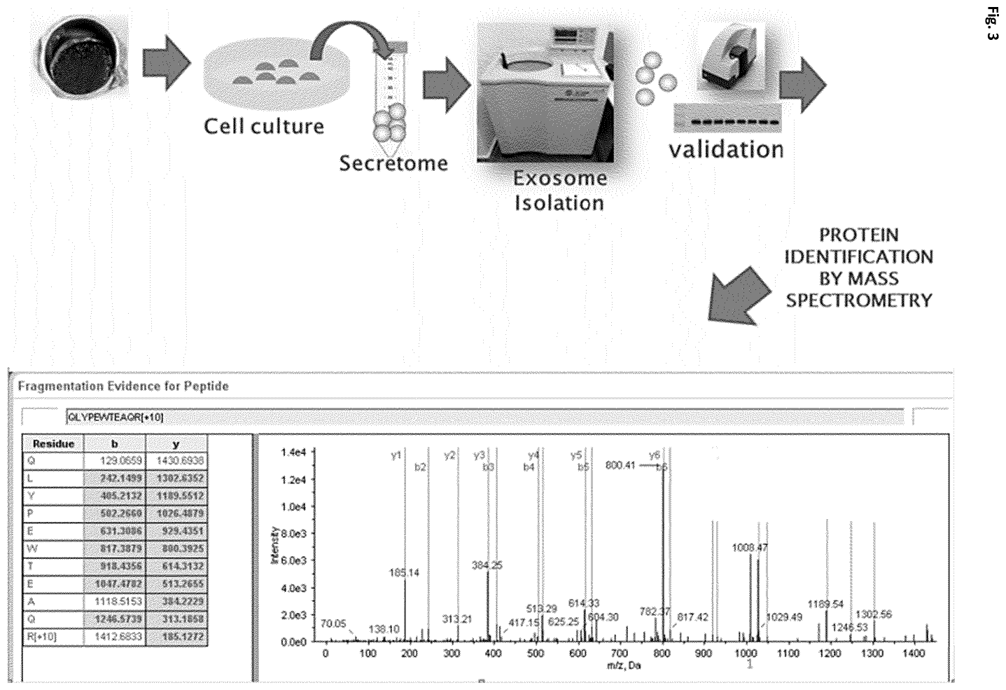

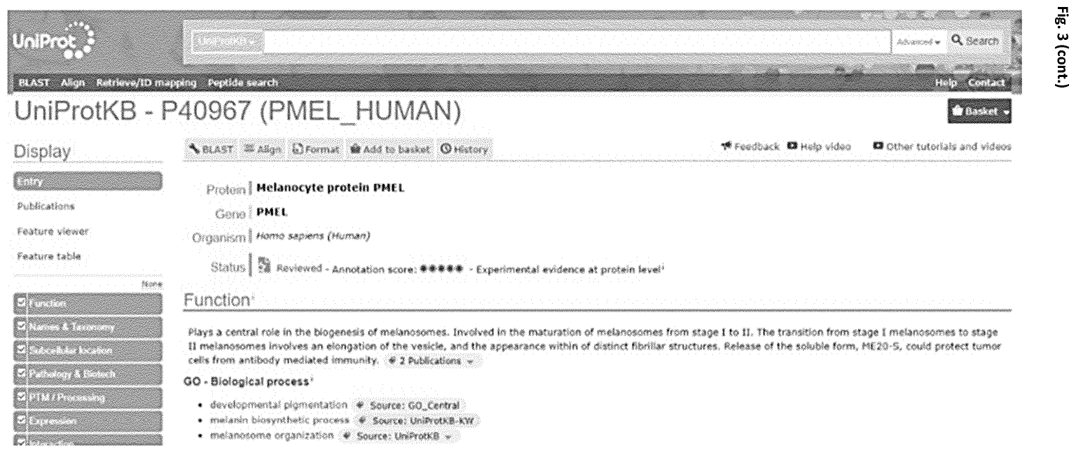

### DETAILED DESCRIPTION OF THE INVENTION

Contrary to other type of cancers, despite that there is currently an efficient diagnosis and treatment of uveal melanoma (UM) tumors, the vital prognosis kept being unaltered for the last decades. Thus, approximately half of the UM diagnosed patients will die of metastatic disease frequently in the liver. One of the most relevant advances on the UM research was the discovery of expression profiles and chromosomal abnormalities that permit, together with other clinical prognostic risk factors, to predict the risk of metastasis at the time of diagnosis classifying the tumors in two main groups: Type 1 with low risk; and Type 2 with high risk of metastasis. However, the study of these alterations at molecular level implies a very invasive procedure that carries its risk and disadvantages.

At the Ocular Oncology Unit in Santiago de Compostela (Spain) we have focused our research on the identification of uveal melanoma non-invasive serum biomarkers for clinical prognostication. Thus, in previous studies (Pardo et al., Proteomics 2005; Int J Cancer 2006; J Proteome Res 2007), the authors of the present invention identified various secreted proteins by uveal melanoma (UM) tumors cells in vitro such as DJ-1/PARK7 and melanocyte protein PMEL/gp100, among others, which were postulated as potential circulating biomarkers as they were subsequently found in the plasma of certain UM patients (Pardo et al., Expert Rev Proteomics 2007). The further assessment of the potential biomarkers led as to identify a positive correlation between serum DJ-1 levels and choroidal nevi risk factors associated to the transformation into uveal melanoma (Bande et al., IOVS 2012). Moreover, we observed an exponential correlation between PMEL/gp100 circulating levels and UM tumour size in UM patients. Interestingly, PMEL/gp100 reached control levels in UM disease free patients, while those with metastasis showed no differences compared to UM. Thus, we suggested the role of DJ-1 oncoprotein as an early nevi malignancy circulating marker and PMEL/gp100 as a predictor of UM tumour growth and probably a guide of clinical staging.

In this sense, the present invention is based on the determination that subjects with uveal melanoma (UM) tumors with metastasis present PMEL/gp100 levels in biological samples much higher than those of control subjects and those that were treated (UM disease free patients), the amount of this protein in the serum of patients was analyzed. The result was an extraordinary increase of melanocyte protein PMEL/ME20-S/gp 100 levels in the serum of patients with uveal melanoma (UM) tumours and those with metastasis compared to the same subjects prior to the metastasis (UM disease free patients) or to control subjects (healthy and nevi).

Furthermore, we suggest that the extraordinary increase in PMEL/ME20-S/gp 100 levels in the serum of patients with uveal melanoma (UM) tumours and metastasis compared to the same subjects prior to the metastasis or to control subjects (UM disease free patients) is due to the fact that PMEL/ME20-S/gp 100 is significantly present in the exosomes shed in particular by uveal melanoma tumor cells. To our knowledge, this is the first time that it is described that PMEL/ME20-S/gp 100 is present in the exosomes from melanoma uveal tumor cells. Such extraordinary increase due to its increased localization in the exosomes facilitates the detection of such biomarker (PMEL/ME20-S/gp 100) in non-invasive samples such as blood or serum biological samples and thus the prognosis of diseases such as metastasis in human subjects suffering or having UM.

Hence, a first aspect of the invention refers to an in vitro method for detecting tumor growth and diagnosing or prognosticating the risk of metastasis in a human subject that has been diagnosed with uveal melanoma; it will also help to monitor systemic treatment efficacy of metastasis. Wherein the method comprises using, as an indicator, the levels of PMEL positive exosomes, obtained from a biological sample isolated from the human subject, preferably selected from the list consisting of blood, or serum, of at least melanocyte protein PMEL/ME20-S/gp 100, and obtaining a result of the method by comparing the expression levels of at least said protein with a reference value or with the expression levels of a control, wherein an increased expression of melanocyte protein PMEL/ME20-S/gp 100 is indicative of a risk of the patient having or suffering from metastasis.

More particularly, the present invention provides an in vitro method for diagnosing tumor growth or prognosticating the risk of metastasis in a human subject that has been diagnosed with uveal melanoma, wherein the biological sample is treated with a lysis agent capable of disrupting the membrane of the exosomes prior to determining the expression level of the melanocyte protein PMEL/ME20-S/gp 100.

More particularly, the present invention provides an in vitro method for diagnosing or prognosticating the risk of metastasis in a human subject that has been diagnosed with uveal melanoma, wherein the biological sample is treated so that the exosomal fraction of the melanoma tumor cells is obtained and the determination of the PMEL/ME20-S/gp 100 levels is performed in such exosomal fraction, wherein such exosomal fraction is treated with a lysis agent, such as a detergent composition or chaotropic agents and/or physical lysis (e.g. Ultrasounds, vigorous shaking), capable of disrupting the membrane of the exosomes prior to determining the expression level of PMEL/ME20-S/gp 100.

In another preferred embodiment of the first aspect of the invention or of any of its preferred embodiments, the biological sample is selected from the group consisting of blood, or serum and such biological sample is treated with a lysis agent such as a detergent composition or chaotropic agents and/or physical lysis (e.g. Ultrasounds, vigorous shaking), capable of disrupting the membrane of the exosomes so that such exosomes, in particular exosomes from melanoma uveal tumor cells, liberate their content to the exterior and thus liberate expression levels of PMEL/ME20-S/gp 100.

In this sense it is noted that collection and handling procedures of blood, serum or even plasma needs to be greatly improved for protein analysis in order to reliably detect differences between healthy and disease patients. Furthermore, there are compounds present in blood that can degrade free proteins. These factors have consequently decreased sensitivity and specificity of protein biomarker assays. Proteins secreted from blood cells into exosomes are protected from degradation and have increased potential as biomarkers. To improve the use of proteins as biomarkers, exosomes have to be either isolated from blood, serum or plasma by different methods and then proteins need to be released from the inside with a lysis agent to disrupt the membrane of the exosomes or simply disrupted without previously isolating them so that they liberate their content to the exterior and thus liberate proteins such as PMEL/ME20-S/gp 100 in the sample (examples of lysis agents useful in the present invention are known to the skilled person). In any case, the fact that this is the first time that it is described that PMEL/ME20-S/gp 100 is upregulated in the exosomes from melanoma uveal tumor cells in the blood, serum or plasma samples of patients suffering from UM, unveils the use of this protein in these samples as a most value tool to reliably detect differences between patients.

In the context of the present invention, the term “detergent composition” is understood as a composition comprising amphipathic molecules (containing both polar hydrophilic heads and non-polar hydrophobic tails) that enables disruption and formation of hydrophobic-hydrophilic interactions among molecules. In biological research, detergents such as oxyethylene, tert-octylphenol or ethyleneglycoether polymers are used to solubilize cell-derived membranes such as plasmatic membranes, organelles membranes or extracellular exosomes in order to allow the release of their content. To not interfiere with soluble proteins n-Octylglucoside or other mild non-denaturing detergent for the solubilization of proteins can be used.

Exosomes are extracellular cell-derived vesicles with a diameter ranging from 30 to 300 nm present in biological fluids and cultured media of cell cultures. They contain proteins, metabolites and nucleic acids such as mRNA and non-coding RNAs coated in a lipid bilayer membrane. The exosomal fraction corresponds to all the exosomes of a particular biological sample or cell culture medium.

In the context of the present invention, the exosomal fraction from melanoma uveal tumor cells can be obtained from any known technique to the skilled person.

A second aspect of the invention, relates to a kit or device comprising at least one or more agents, such as antibodies or fragments thereof, capable of detecting melanocyte protein PMEL/ME20-S/gp 100, means for detecting said protein in the biological sample and a lysis agent capable of disrupting the membrane of exosomes.

A third aspect of the invention, refers to an In vitro use of the kit according to or as defined in the second aspect of the invention, for diagnosing or prognosticating the risk of metastasis in a human subject that has been diagnosed with uveal melanoma by using a serum or blood sample obtained from said subject.

Preferably, said kit or device additionally comprises means capable of detecting melanocyte protein PMEL/ME20-S/gp 100 immobilized on a surface, preferably on the surface of a microarray.

It is noted that Bande M F, Santiago M, Mera P, et al. ME20-S as a potential biomarker for the evaluation of uveal melanoma. Invest Ophthalmol Vis Sci. 2015; 56:7007-7011. DOI:10.1167/iovs.15-17183, already demonstrated for the first time a positive correlation between serum ME20-S protein levels and UM tumor thickness. In addition, said document demonstrated that while those patients with treated UM (DFUM) showed no significant differences compared to healthy individuals, increased serum ME20-S levels are positively associated with the presence of nontreated UM and the existence of UM metastatic disease.

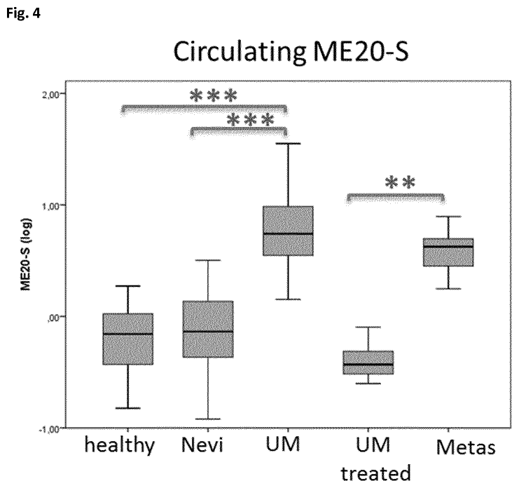

The present invention, as shown in FIG. 4, further develops this work by demonstrating that the extraordinary increase in PMEL/ME20-S/gp 100 levels in the serum of patients with uveal melanoma (UM) tumours and metastasis compared to the same subjects prior to the metastasis or to control subjects (UM disease free patients) is due to the fact that PMEL/ME20-S/gp 100 is significantly present in the exosomes shed in particular by uveal melanoma tumor cells.

In this sense, FIG. 4 provides the following facts: 1. ME20-S circulating levels are statistically elevated in patients with primary UM tumors; 2. The ME20-S circulating levels positively correlate with tumor size; 3. ME20-S levels decrease to control levels in those patients were their primary tumor was treated by braquitherapy/enucleation (UM disease free patients); 4. ME20-S levels are statistically elevated in those patients with systemic disease. Therefore, in this invention we suggest the quantification of PMEL-positive exosomes liberated into the circulation as a specific UM biomarker to assay: a) primary tumor growth, b) tumor risk of metastasis, c) early systemic dissemination (before any scan) and d) systemic therapy follow up (chemotherapy/immunotherapy).

1. An in vitro method for detecting tumor growth and diagnosing or prognosticating the risk of metastasis in a human subject that has been diagnosed with uveal melanoma, wherein the method comprises using, as an indicator, the expression levels, obtained from a biological sample isolated from the human subject, preferably selected from the list consisting of blood, or serum, of at least melanocyte protein PMEL/ME20-S/gp 100 and obtaining a result of the method by comparing the expression levels of at least said protein with a reference value or with the expression levels of a control, wherein an increased expression of melanocyte protein PMEL/ME20-S/gp 100 is indicative of a risk of the patient having or suffering from metastasis.

2. The method according to claim 1, wherein the biological sample is treated with a lysis agent capable of disrupting the membrane of the exosomes prior to determining the expression level of the melanocyte protein PMEL/ME20-S/gp 100.

3. The method according to claim 1, wherein the biological sample is treated so that the exosomal fraction of the melanoma tumour cells is obtained and the determination of the expression levels of PMEL/ME20-S/gp 100 is performed in such exosomal fraction, wherein such exosomal fraction is treated with a lysis agent capable of disrupting the membrane of the exosomes prior to determining the expression level of PMEL/ME20-S/gp 100.

4. A kit or device comprising at least one or more agents, such as antibodies or fragments thereof, capable of detecting melanocyte protein PMEL/ME20-S/gp 100, means for detecting said protein in the biological sample and a lysis agent capable of disrupting the membrane of exosomes.

5. In vitro use of the kit according to claim 4, for detecting tumor growth and diagnosing or prognosticating the risk of metastasis in a human subject that has been diagnosed with uveal melanoma by using a serum or blood sample obtained from said subject.
